(19) United States
(12) Patent Application Publication
(10) Pub. No.: US 2005/0149189 A1
(43) Pub. Date: Jul. 7, 2005

(54) Intervertebral disk prosthesis

(76) Inventors: Mourad Ben Mokhtar, Paris (FR); Olivier Carli, Geneva (CH)

Correspondence Address: Carl M. Napolitano, Ph.D.;Allen, Dyer, Doppelt, Milbrath & Gilchrist, P.A., Suite 1401, 255 South Orange Avenue, Orlando, FL, 32801, US

(21) Appl. No.: 10/865,487

(22) Filed: Jun. 10, 2004

(30) Foreign Application Priority Data
Jan. 7, 2004 (FR) 04 00087

(51) Int. Cl. A61F 2/44

(52) U.S. Cl. 623/17.11

(57) ABSTRACT

A cervical disk prosthesis includes a ball and socket joint interposed between opposing plates for attaching to adjacent vertebrae. The bearing plate has a dimension in the frontal plane larger than a corresponding dimension of the upper plate for providing a limit stop for the upper plate, and by its outside face, a bearing surface for a vertebra that is greater than that presented by the second plate. The disk prosthesis has a trapezoidal form in the horizontal plane with the upper and smaller plate having a parallelepiped shape for defining flexion and extension mobility. One of the plates carries an elastic ring about the perimeter for making a shock absorbing contact with the opposing plate.

### CROSS REFERENCE TO RELATED APPLICATION

This application incorporates by reference and claims priority to French Application having Registration Number 04 00087 filed on Jan. 7, 2004 for “Discal Prosthesis For Vertebrae”.

### FIELD OF INVENTION

The present invention generally relates to spinal implants for use in intervertebral disk replacement, and more particularly to an articulating disk prostheses and insertion device for artificially replacing the fibro-cartilaginous disk that connects vertebrae of a spinal column.

### BACKGROUND

An intervertebral disk comprising a deformable element, known as nucleus pulposus, surrounded by a number of elastic fibrous layers, can undergo alterations such as compression, deformation, slippage or wear and, more generally, degeneration associated with mechanical stresses applied to it. This may result in anatomical and functional destruction of the disk and of the vertebral segment. This alteration of the disk changes the mechanical behavior of the disk and leads to a reduction in the height of the intersomatic space, which results in a disturbance of articular function as a whole. This produces instability that, in particular, creates an osteoarthritic reaction that is the source of pain and of osteophytic processes.

It is well known to replace a defective disk with an artificial disk, designed so as to attempt to reproduce the kinematics of a natural movement. For example, U.S. Pat. No. 5,562,738 describes a disk prosthesis including first and second plates made of a metallic material such as titanium for attachment to adjacent vertebrae. A ball-and-socket joint between the plates comprises a first insert mounted on one of the plates and a spherical cap cooperating with the spherical cupola of a second insert mounted on the other plate. The inserts are made of a biocompatible ceramic material with improved tribological characteristics, specifically with respect to its wear resistance.

However, such a disk prosthesis does not allow the vertebrae to regain their natural mobility. Indeed, such prosthesis has limited clearances in certain planes that depend on asymmetric forms of embodiments of a ball-and-socket joint. The ball-and-socket joint has a form that is relatively difficult to handle and is sensitive to breakage or cracking, thus reducing the life span of the prosthesis.

In order to develop a prosthesis for reproducing the kinematics of the natural movement, patent FR 2 730 159 describes a prosthesis involving an intermediate core with two spherical faces oriented in the same direction but having different radii. Such an intermediate nucleus is designed to slide over a convex surface belonging to a lower plate. The core also has a convex upper surface on which the upper plate slides. The core thus has mobility in the horizontal plane, so that it is capable of moving to one side when the plates approach from the opposing side. Such a prosthesis, however, has the disadvantage of ejecting the core toward the outside of the prosthesis.

In order to prevent ejection of the core from the prosthesis, French Patent FR 2 659 226 describes a prosthesis whose upper plate has a concave face that slides on a polyethylene core in the form of a spherical cap, fixed securely in a hollow of a lower plate. Lower and upper titanium plates are each equipped with bosses for anchoring in a respective vertebra to avoid risk of separation. Double baffle flanges limit the angular clearance of the lower and upper plates using complementary sections on the circumference of the upper plate and on the circumference of the lower plate. In practice, the double baffle flanges are intended to overlap at the moment of maximum angular shifting between the plates, which can lead to an undesirable nesting with friction, even blockage. Moreover, such prosthesis requires a relatively traumatizing implantation in the vertebral plates and does not make it possible to ensure the optimum transfer of the thrust of the upper vertebra on the lower vertebra. Further, the contact interface between a polyethylene core and a titanium plate degrades over time, thus undesirably modifying the mobility of the prosthesis.

The present invention remedies such drawbacks by providing an intervertebral disk prosthesis for vertebrae that faithfully reproduces the natural movements of the disk and ensures an optimum transmission of the thrust of upper vertebra on lower vertebra while offering control of the relative angular clearance between the vertebrae.

### SUMMARY

A spinal disk prosthesis restores normal physiological function in the spine by preserving intervertebral motion, stability, lordosis, and spacing while protecting vascular, neural, and other spinal structures.

One embodiment of the present invention provides a prosthesis that may include first and second plates for attaching to adjacent vertebrae, and a ball-and-socket joint interposed between the two plates mounted in stacked form one on top of the other, so that the inside faces of these plates are turned toward one another. The joint may comprise a spherical cap fitting into a spherical cupola. The first plate, herein referred to as a bearing plate, has a dimension in the frontal plane larger than the corresponding dimension of the second plate for providing a limit stop for the second plate, and by its outside face, a bearing surface for a vertebra that is greater than that presented by the second plate.

The disk prosthesis may further have a substantially trapezoidal form in the horizontal plane, with its large base delimiting the leading edge of the plate while the small base delimits the training edge. The second plate may have a parallelepiped shape in the horizontal plane.

The plates may be dimensioned in such a way that: E/d=ED₁ for A₁/A₂>0.5, with E: distance between the two inner faces of the plates; d: width of the upper plate in the frontal plane; D₁: length of the upper plate in the sagittal plane; A₁: maximum angle of clearance between the plates in the frontal plane; and A₂: maximum angle of clearance between the plates in the sagittal plane.

The first plate may have an inner face with a flat profile in the frontal plane and an outer face with a convex profile in the frontal plane. One of the plates may include, on its inner face, an elastic ring that makes contact with the inner face of the other plate.

### BRIEF DESCRIPTION OF DRAWINGS

For a fuller understanding of the invention, reference is made to the following detailed description, taken in connection with the accompanying drawings illustrating various embodiments of the present invention, in which:

FIG. 1 is a front view in the frontal plane of a first exemplified embodiment of prosthesis according to the invention in a median position;

FIG. 2 is a cutaway view in the frontal plane of the prosthesis according to the invention, in an inclined position;

FIG. 3 is a side view in the sagittal plane of the prosthesis according to the invention in a median position;

FIG. 4 is a cutaway view in the sagittal plane of prosthesis according to the invention in an inclined position;

FIG. 5 is a top perspective in the horizontal or coronal plane of the prosthesis according to the invention;

FIGS. 6 and 7 are cutaway views in the frontal plane and in the sagittal plane, respectively, of a second exemplified embodiment of prosthesis according to the invention in an inclined position;

FIGS. 8 and 9 are cutaway views in the frontal plane and in the sagittal plane, respectively, of a third exemplified embodiment of the prosthesis according to the invention in an inclined position;

FIGS. 10 and 11 are front and side views of an alternate embodiment of the disk prosthesis of FIG. 1;

FIG. 12 is a cross sectional view taken through lines 12-12 of FIG. 10;

FIG. 12A is a partial exploded view of a shock absorbing portion of FIG. 12;

FIGS. 13 and 14 are front and side views of an alternate embodiment of the disk prosthesis of FIG. 10; and

FIG. 15 is a cross sectional view taken through lines 15-15 of FIG. 13.

### DETAILED DESCRIPTION OF EMBODIMENTS

The present invention will now be described more fully with reference to the accompanying drawings in which alternate embodiments of the invention are shown and described. It is to be understood that the invention may be embodied in many different forms and should not be construed as limited to the illustrated embodiments set forth herein. Rather, these embodiments are provided so that this disclosure may be thorough and complete, and will convey the scope of the invention to those skilled in the art.

With reference initially to FIGS. 1-4, one embodiment of the present invention may be described as a disk prosthesis 10 to be implanted in place of a intervertebral disk between two adjacent vertebrae 12, 14. One embodiment of the disk prosthesis 10 herein described, by way of example, includes a first plate 16 illustrated as a lower plate in the illustrated example, and a second plate 18 illustrated as an upper plate. The plates 16, 18 are intended to be attached to the adjacent vertebrae 12, 14 and are herein described, by way of example, as each having an outer face, 20, 22, respectively, and each having an inner face 24, 26, respectively, extending opposite one another. Embodiments of the first and second plates 16, 18, as herein described by way of example, may be formed from a biometallic material, and optionally have an asteo-conductive coating. By way of example, the plates 16, 18 may be made of titanium or a titanium alloy. In one embodiment, the first and second plates 16, 18 comprise titanium with an hydroxy-apatite coating.

With continued reference to FIG. 1, and to FIG. 5, the first plate 16, also known as a carrier plate, has a transversal dimension D greater than the corresponding dimension d of the second plate 18. These dimensions d, D are measured in a transversal direction at the intersection between the horizontal plane T, illustrated with reference again to FIG. 5, and the frontal plane F, illustrated with reference again to FIG. 1, and are herein considered in terms of well known anatomical planes.

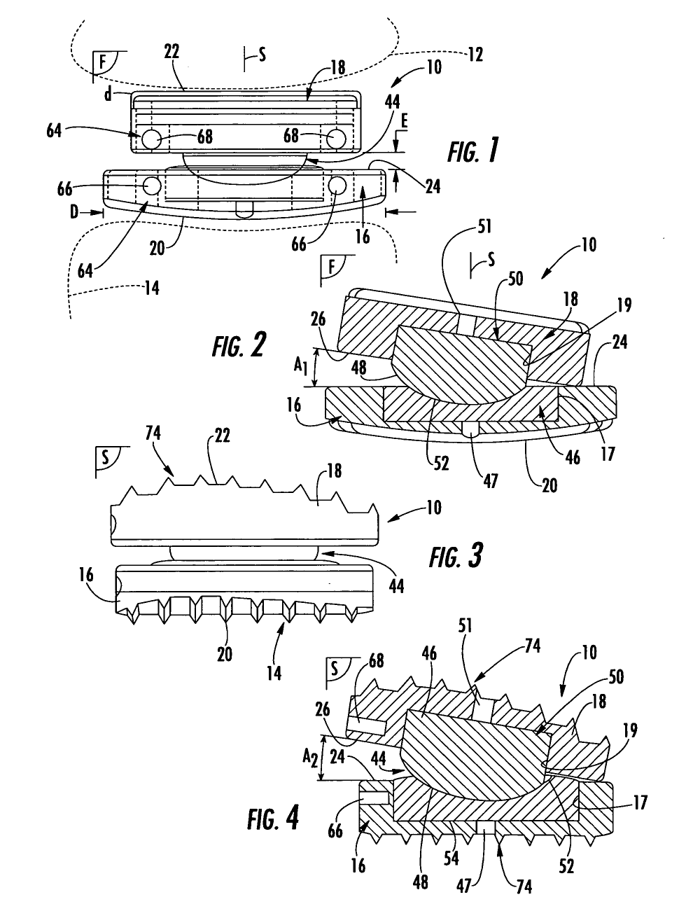

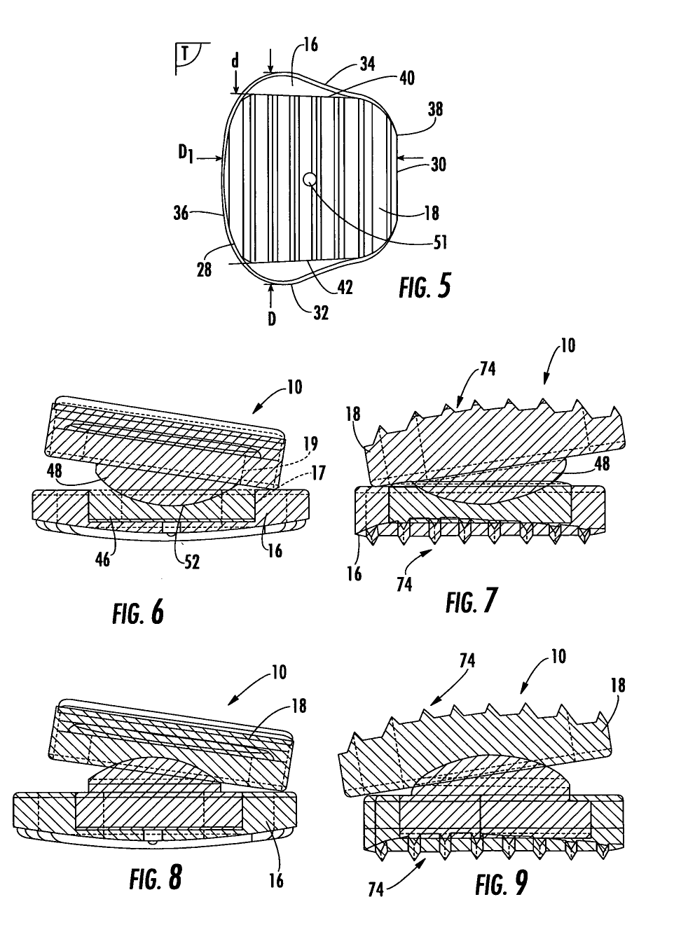

With reference to FIGS. 2 and 4, the inner face 24 of the first plate 16 thus constitutes a limit stop for the second plate 18, and with the outer face 20 of the first plate 16, a bearing surface for the vertebra 14 that is larger than the surface of the vertebra 12 contacted by the outer surface 22 presented by the second plate 18. As illustrated with reference again to FIG. 5, the first plate 16, the bearing plate, has in the horizontal plane T, a substantially trapezoidal shape whose large base defines a leading edge 28, while a small base defines a trailing edge 30. The training edge 30 is connected to the leading edge 28 by opposing divergent edges 32, 34. Connecting fillets may preferably connect the leading and trailing edges 28, 30 to the divergent edges 32, 34.

As illustrated with reference again to FIGS. 1 and 2 for one embodiment herein described by way of example, the first plate 16 has the inner face 24 with a flat profile and the outer face 20 with a convex profile in the frontal plane F. With reference again to FIG. 5, the second plate 18 is, in the horizontal plane T, substantially the shape of a parallelepiped, with a leading edge 36 and a trailing edge 38 that are substantially parallel and merged in a stacked position with the leading edge 28 and the trailing edge 30 of the first plate 16. The leading edge 36 of the second plate 18 is connected to the trailing edge 38 through two linking edges 40, 42 substantially parallel to one another. Connecting fillets may connect the leading and trailing edges 36, 38 to the linking edges 40, 42. As is clearly shown with reference again to FIGS. 1, 2, and 5, the first plate 16 thus juts out in the horizontal plane T on either side of the linking edges 40, 42 of the second plate 18.

With continued reference to FIGS. 1-4, the disk prosthesis 10 herein described includes a ball-and-socket joint 44 interposed between the two plates 16, 18 that are stacked one on top of the other. In the exemplified embodiment illustrated in FIGS. 1 to 5, the ball-and-socket joint 44 comprises cooperating arcuate surfaces including a first insert 46 that has a spherical cap 48 and a second insert 50 that has a spherical cup 52 that smoothly works with the spherical cap 48. Each insert 48, 50 is mounted in a hole 54, 56, a blind hole for example, through the inner face 24, 26 of each plate 16, 18. Each insert 46, 50 has a general form of revolution and has, respectively, a base 58, 60 of circular transversal cross-section, one of whose ends is arranged so as to have either the spherical cap 48 or the spherical cup 52. In one embodiment, the inserts 46, 50 are made of a ceramic material or of polyethylene.

As illustrated with reference again to FIGS. 2 and 4, by way of example, vent holes 47, 51 are provided within the first and second plates 16, 18, respectively, and extend from cavities 17, 19 formed within the first and second plates 16, 18. The vent holes 47, 51 permit air to escape therethrough when inserting the inserts 46, 50 into cavities 17, 19 during a manufacturing process.

It is clear from the preceding description that the first plate 16, the carrier plate, is wider than the second plate 18 in the transverse direction such as to constitute, on the one hand, the flat limiting surface, the inner surface 24, for the second plate 18 and, on the other hand, a large bearing surface, the outer face 20, with the associated vertebra. As is further clear with reference again to FIGS. 2 and 4, the prosthesis limit positions, both in the frontal plane F and in the sagittal plane S, are determined by bringing the plates 16, 18 into contact with one another.

As further illustrated with reference again to FIGS. 1-5, one embodiment of the disk prosthesis 10 may be dimensioned in such a manner that: E/d=ED₁ for A₁/A₂>0.5, wherein E represents the distance between the two inner faces 24, 26 of the plates 16, 18; d represents the width of the second (upper) plate 18 in the frontal plane F; D₁ represents the length of the second (upper) plate 18 in the sagittal plane S; A₁ represents the maximum angle of clearance between the plates 16, 18 in the frontal plane F; and A₂ represents the maximum angle of clearance between plates 16, 18 in the sagittal plane S. For the example of the disk prosthesis 10, herein described by way of example, the angles A are about ten degrees or less but may be larger as desired for a particular use without deviating from the teaching of et present invention. Such dimensioning makes it possible to provide a disk prosthesis with controlled clearances that correspond substantially to the natural movements of the spinal disk.

In the example illustrated with reference to FIGS. 1 to 5, the spherical cap 48 and the spherical cup 52 are created on the inserts 46, 50 attached to the plates 16, 18. Alternatively, and as illustrated with reference to FIGS. 6 and 7, another variant embodiment in which the spherical cap 48 is an integral part of the second plate 18, the upper plate in the example illustrated. For this embodiment, the spherical cap 48 may be made of titanium, while the spherical cup 52 is arranged in an insert 46 made of ceramic or polyethylene.

Further, in the preceding examples, the spherical cap 48 is carried on the second (upper) plate 18 with the spherical cup 52 carried by the first (lower) plate 16. Alternatively, and with reference to FIGS. 8 and 9, another variant embodiment may include the spherical cup 52 carried by the second (upper) plate 18, with the spherical cap 48 carried by the first (lower) plate 16. Yet further and by way of example, one embodiment may include a zirconia-on-alumina articulating surface for the spherical cap 48 and the spherical cup 52 of the ball and socket joint 44.

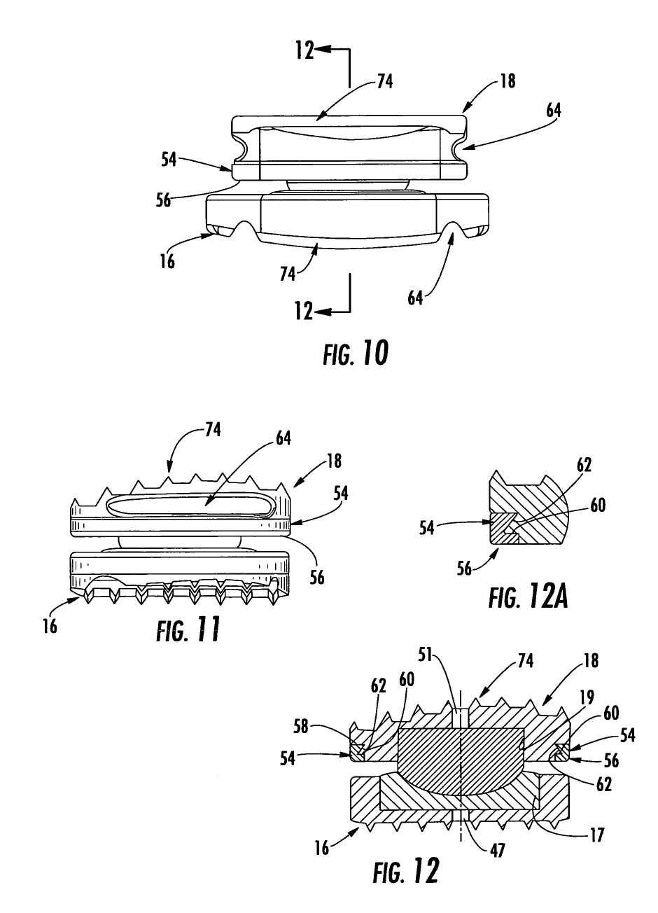

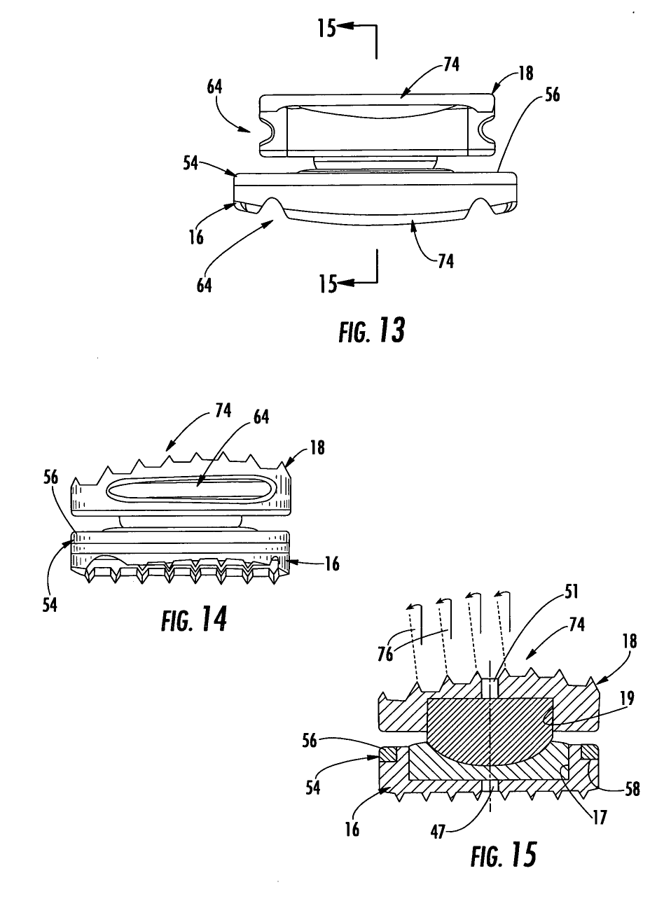

Yet further, and with reference to FIG. 10-15, by way of example, either the first plate 16 or the second plate 18 may carry a shock absorbing material 54 for contacting the opposing plate 16, 18 at the limits of movement for the articulating plates. As herein presented by way of example, the absorbing material may be an elastic ring 56 extending about and carried in frictional contact within the perimeter portions of the inner faces 24, 26 of the plates 16, 18. As herein illustrated by way of example, the elastic ring 62 may be mounted in a recess 58 surrounding the spherical cap 48 and/or the spherical cup 52 and arranged on the inner face 24, 26. In one embodiment, the plates 16, 18 may include a notch or recess 58 extending about the perimeter fro receiving the elastic ring 56 therein. Yet further, and as illustrated with reference to FIG. 12, the elastic ring 56 may include an inner groove 60 for receiving a tab 62 within a wall of the recess 58 for retaining the elastic ring 56 within the recess 58.

As illustrated with reference again to FIGS. 1 and 4, one embodiment of the disk prosthesis 10 may comprise positioning cutouts 64, such as the holes 66 within the anterior surface of the first plate 16 and holes 68 carried within the second plate 18 for receiving a tool for simultaneously holding the plates. Alternatively, and as illustrated with reference to FIGS. 10, 11, 13, and 14, the cutouts 64 may comprise a pair of parallel groves 70, 72 carried by each of the first and second plates 16, 18 respectively, for receiving the tool. By way of further example, and with continued reference to FIGS. 10-15, the pairs of parallel groves 70, 72 for the first and second plates 16, 18 may extend from the anterior to the posterior portions thereof, with the first plate including the grooves 70 along the outer face 20 and the second plate 18 including the grooves 72 along opposing side wall surfaces thereof.

For the above described embodiments of the disk prosthesis 10, the outer faces 20, 22 for at least one of the first and second plates 16, 18 comprises a plurality of teeth 74 for retaining the plates between the adjacent vertebrae 12, 14. As illustrated by way of example with reference again to FIG. 5, at least a portion of the plurality of teeth may comprise an anterior slope bias for facilitating an insertion into a space between vertebrae while restricting anterior distraction of the plates 16, 18.

Many modifications and other embodiments of the invention will come to the mind of one skilled in the art having the benefit of the teachings presented in the foregoing descriptions and the associated drawings. Therefore, it is to be understood that the invention is not to be limited to the specific embodiments disclosed, and that modifications and alternate embodiments are intended to be included within the scope of the appended claims.

1. A disk prosthesis comprising:
a first plate for attaching to a first vertebrae;
a second plate for attaching to a second adjacent vertebrae, wherein a transversal dimension of the first plate is greater than a corresponding dimension of the second plate, so as to constitute, by the inner face of the first plate, a limit stop for the second plate and, by the outer face of the first plate, a bearing surface for the vertebrae that is larger than a bearing surface for the outer face of the second plate; and
a ball and socket joint interposed between the first and second plates stacked one on top of the other, so that inner faces of the plates are turned toward one another, wherein a ball portion of the ball and socket joint comprises a spherical cap cooperating with a socket portion of the joint comprising a spherical cup.

2. A disk prosthesis according to claim 1, wherein first plate comprises a substantially trapezoidal shape in a horizontal plane, and wherein a large base of the first plate defines a leading edge and a small base thereof defines a trailing edge.

3. A disk prosthesis according to claims 1, wherein the second plate is substantially shaped as a parallelepiped in the horizontal plane.

4. A disk prosthesis according to claim 1, wherein the first and second plates are dimensioned such that E/d is at least approximately equal to E/D₁ for A₁/A₂>0.5, wherein E represents a distance between the two inner faces of the first and second plates for a parallel positioning thereof, d represents a width of the second plate in the frontal plane, D₁ represents a length of the second plate in the sagittal plane, A₁ represents a maximum angle of clearance between the first and second plates in the frontal plane, and A₂ represents a maximum angle of clearance between the first and second plates in the sagittal plane.

5. A disk prosthesis according to claim 4, wherein at least one of A₁ and A₂ does not exceed ten degrees.

6. A disk prosthesis according to claim 1, wherein the first plate has an inner face having a flat profile in a frontal plane and an outer face having a convex profile in the frontal plane.

7. A disk prosthesis according to claim 1, wherein the first and second plates comprise a biometallic material having an osteoconductive coating.

8. A disk prosthesis according to claim 1, wherein articulating surfaces of the spherical cap and the spherical cup for the ball and socket joint comprise at least one of a zirconia-on-alumina, zirconia-on-zirconia, and alumina-on-alumina material.

9. A disk prosthesis according to claim 1, wherein the spherical cap comprises a titanium material and the spherical cup comprises at least one of a ceramic material and a polyethylene material.

10. A disk prosthesis according to claim 1, wherein at least one of the first and second plates carries a shock absorbing medium on the inner face thereof for making contact with the at least one of the first and second opposing plates.

11. A disk prosthesis according to claim 10, wherein the shock absorbing medium comprises an elastic ring extending about a perimeter of the at least one of the first and second plates.

12. A disk prosthesis according to claim 10, further comprising a recess within at least one of the first and second plates for carrying the shock absorbing medium therein.

13. A disk prosthesis according to claim 1, further comprising at least one positioning cutout carried within each of the first and second plates for receiving a tool for simultaneously holding the plates thereby.

14. A disk prosthesis according to claim 13, wherein the at least one positioning cutout comprises a hole carried within anterior portions for each of the first and second plates.

15. A distal prostheses according to claim 13, wherein the at least one positioning cutout for each of the first and second plates comprises a pair of parallel grooves positioned for receiving a tool for simultaneously holding the plates thereby.

16. A distal prosthesis according to claim 15, wherein the pair of parallel grooves extends from anterior to posterior portions of the first and second plates, and wherein the first plate includes the grooves along the outer face and the second plate includes the grooves along opposing side wall surfaces thereof.

17. A distal prosthesis according to claim 1, wherein the outer face for at least one of the first and second plates comprises a plurality of teeth for retaining the plates between the adjacent vertebrae.

18. A distal prosthesis according to claim 17, wherein at least a portion of the plurality of teeth comprises an anterior slope bias for facilitating an insertion into a space between the first and second vertebrae while restricting anterior distraction of the plates therefrom.

19. A disk prosthesis comprising:
a first plate comprises a substantially trapezoidal shape in a horizontal plane for attaching to a first vertebrae;
a second plate shaped as a parallelepiped in the horizontal plane for attaching to a second adjacent vertebrae; and
a ball and socket joint interposed between the first and second plates stacked one on top of the other, so that inner faces of the first and second plates face one another, wherein a ball portion of the ball and socket joint comprises a spherical cap cooperating with a socket portion of the joint comprising a spherical cup.

20. A disk prosthesis according to claim 19, wherein a transversal dimension of the first plate is greater than a corresponding dimension of the second plate, so as to constitute, by the inner face of the first plate, a limit stop for the second plate and, by the outer face of the first plate, a bearing surface for the vertebrae that is larger than a bearing surface for the outer face of the second plate.

21. A disk prosthesis according to claim 19, wherein the first and second plates are dimensioned such that E/d is at least approximately equal to E/D₁ for A₁/A₂>0.5, wherein E represents a distance between the two inner faces of the first and second plates for a parallel positioning thereof, d represents a width of the second plate in the frontal plane, D₁ represents a length of the second plate in the sagittal plane, A₁ represents a maximum angle of clearance between the first and second plates in the frontal plane, and A₂ represents a maximum angle of clearance between the first and second plates in the sagittal plane.

22. A disk prosthesis according to claim 21, wherein at least one of A₁ and A₂ does not exceed ten degrees.

23. A disk prosthesis according to claim 19, wherein the first plate has an inner face having a flat profile in a frontal plane and an outer face having a convex profile in the frontal plane.

24. A disk prosthesis according to claim 19, wherein at least one of the first and second plates carries a shock absorbing medium on the inner face thereof for making contact with the at least one of the first and second opposing plate.

25. A disk prosthesis according to claim 24, wherein the shock absorbing medium comprises an elastic ring extending about a perimeter of the at least one of the first and second plates.

26. A disk prosthesis according to claim 24, further comprising a recess within at least one of the first and second plates for carrying the shock absorbing medium therein.

27. A disk prosthesis according to claim 19, further comprising at least one positioning cutout carried within each of the first and second plates for receiving a tool for simultaneously holding the plates thereby.

28. A disk prosthesis according to claim 27, wherein the at least one positioning cutout comprises a hole carried within anterior portions for each of the first and second plates.

29. A distal prostheses according to claim 27, wherein the at least one positioning cutout for each of the first and second plates comprises a pair of parallel grooves positioned for receiving a tool for simultaneously holding the plates thereby.

30. A distal prosthesis according to claim 29, wherein the pair of parallel grooves extends from anterior to posterior portions of the first and second plates, and wherein the first plate includes the grooves along the outer face and the second plate includes the grooves along opposing side wall surfaces thereof.

31. A disk prosthesis comprising a first plate and an opposing second plate operable therebetween by a ball and socket joint, wherein the first and second plates are dimensioned such that E/d is at least approximately equal to E/D₁ for A₁/A₂>0.5, wherein E represents a distance between the two inner faces of the first and second plates for a parallel positioning thereof, d represents a width of the second plate in the frontal plane, D₁ represents a length of the second plate in the sagittal plane, A₁ represents a maximum angle of clearance between the first and second plates in the frontal plane, and A₂ represents a maximum angle of clearance between the first and second plates in the sagittal plane.

32. A disk prosthesis according to claim 31, wherein at least one of A₁ and A₂ does not exceed ten degrees.

33. A disk prosthesis according to claim 31, wherein the first plate comprises a substantially trapezoidal shape in a horizontal plane and the second plate is substantially shaped as a parallelepiped in the horizontal plane.

34. A disk prosthesis according to claim 31, wherein a transversal dimension of the first plate is greater than a corresponding dimension of the second plate, so as to constitute, by the inner face of the first plate, a limit stop for the second plate and, by the outer face of the first plate, a bearing surface for the vertebrae that is larger than a bearing surface for the outer face of the second plate.

35. A disk prosthesis according to claim 31, wherein at least one of the first and second plates carries a shock absorbing medium on the inner face thereof for making contact with the at least one of the first and second opposing plate.

36. A disk prosthesis according to claim 35, wherein the shock absorbing medium comprises an elastic ring extending about a perimeter of the at least one of the first and second plates.

37. A disk prosthesis according to claim 35, wherein at least one of the first and second plates comprises a recess for carrying the shock absorbing medium therein.

38. A disk prosthesis according to claim 31, further comprising at least one positioning cutout carried within each of the first and second plates for receiving a tool for simultaneously holding the plates thereby.

39. A disk prosthesis according to claim 38, wherein the at least one positioning cutout comprises a hole carried within anterior portions for each of the first and second plates.

40. A distal prostheses according to claim 38, wherein the at least one positioning cutout for each of the first and second plates comprises a pair of parallel grooves positioned for receiving a tool for simultaneously holding the plates thereby.

41. A distal prosthesis according to claim 40, wherein the pair of parallel grooves extends from anterior to posterior portions of the first and second plates, and wherein the first plate includes the grooves along the outer face and the second plate includes the grooves along opposing side wall surfaces thereof.

42. A disk prosthesis comprising:
a first plate for attaching to a first vertebrae;
a second plate for attaching to an adjacent second vertebrae;
a ball and socket joint interposed between the first and second plates stacked one on top of the other, so that inner faces of the first and second plates are turned toward one another; and
a shock absorbing medium carried by at least one of the first and second plates for making contact with the at least one of the first and second opposing plates.

43. A disk prosthesis according to claim 42, wherein the shock absorbing medium comprises an elastic ring extending about a perimeter of the at least one of the first and second plates.

44. A disk prosthesis according to claim 43, further comprising a recess within at least one of the first and second plates for carrying the shock absorbing medium therein.

45. A disk prosthesis according to claim 42, wherein a transversal dimension of the first plate is greater than a corresponding dimension of the second plate, so as to constitute, by the inner face of the first plate, a limit stop for the second plate, wherein the shock absorbing medium is positioned at the limit stop.

46. A disk prosthesis according to claim 42 wherein the first plate comprises a substantially trapezoidal shape in a horizontal plane and the second plate is substantially shaped as a parallelepiped in the horizontal plane.

47. A disk prosthesis according to claim 42 wherein the first and second plates are dimensioned such that E/d is at least approximately equal to E/D₁ for A₁/A₂>0.5, wherein E represents a distance between the two inner faces of the first and second plates for a parallel positioning thereof, d represents a width of the second plate in the frontal plane, D₁ represents a length of the second plate in the sagittal plane, A₁ represents a maximum angle of clearance between the first and second plates in the frontal plane, and A₂ represents a maximum angle of clearance between the first and second plates in the sagittal plane.

48. A disk prosthesis according to claim 47, wherein at least one of A₁ and A₂ does not exceed ten degrees.

49. A disk prosthesis according to claim 42, further comprising at least one positioning cutout carried within each of the first and second plates for receiving a tool for simultaneously holding the plates thereby.

50. A disk prosthesis according to claim 49, wherein the at least one positioning cutout comprises a hole carried within anterior portions for each of the first and second plates.

51. A distal prostheses according to claim 49, wherein the at least one positioning cutout for each of the first and second plates comprises a pair of parallel grooves positioned for receiving a tool for simultaneously holding the plates thereby.

52. A distal prosthesis according to claim 51, wherein the pair of parallel grooves extends from anterior to posterior portions of the first and second plates, and wherein the first plate includes the grooves along the outer face and the second plate includes the grooves along opposing side wall surfaces thereof.

53. A disk prosthesis comprising:
a first plate for attaching to a first vertebrae;
a second plate for attaching to an adjacent second vertebrae, wherein at least one groove is formed within each of the first and second plates for receiving a tool for frictionally holding the first and second plates thereby; and
a ball and socket joint interposed between the first and second plates stacked one on top of the other, so that inner faces of the first and second plates are turned toward one another.

54. A disk prosthesis according to claim 53, wherein the at least one groove comprises a pair of parallel grooves.

55. A disk prosthesis according to claim 54, wherein the first plate includes the grooves along the outer face and the second plate includes the grooves along opposing side wall surfaces thereof.

56. A disk prosthesis according to claim 55, wherein the pair of parallel grooves extends from anterior to posterior portions of the first and second plates.

57. A disk prosthesis according to claim 53, further comprising a shock absorbing medium carried by at least one of the first and second plates for making contact with the at least one of the first and second opposing plates.

58. A disk prosthesis according to claim 57, wherein the shock absorbing medium comprises an elastic ring extending about a perimeter of the at least one of the first and second plates.

59. A disk prosthesis according to claim 57, further comprising a recess within at least one of the first and second plates for carrying the shock absorbing medium therein.

60. A disk prosthesis according to claim 53, wherein a transversal dimension of the first plate is greater than a corresponding dimension of the second plate, so as to constitute, by the inner face of the first plate, a limit stop for the second plate.

61. A disk prosthesis according to claim 53 wherein the first plate comprises a substantially trapezoidal shape in a horizontal plane and the second plate is substantially shaped as a parallelepiped in the horizontal plane.

62. A disk prosthesis according to claim 53 wherein the first and second plates are dimensioned such that E/d is at least approximately equal to E/D₁ for A₁/A₂>0.5, wherein E represents a distance between the two inner faces of the first and second plates for a parallel positioning thereof, d represents a width of the second plate in the frontal plane, D₁ represents a length of the second plate in the sagittal plane, A₁ represents a maximum angle of clearance between the first and second plates in the frontal plane, and A₂ represents a maximum angle of clearance between the first and second plates in the sagittal plane.

63. A disk prosthesis according to claim 62, wherein at least one of A₁ and A₂ does not exceed ten degrees.
